(19) United States
(12) Patent Application Publication
(10) Pub. No.: US 2023/0282310 A1
(43) Pub. Date: Sep. 7, 2023

(54) Microorganism Discrimination Method and System

(71) Applicant: SHIMADZU CORPORATION, Kyoto-shi (JP)

(72) Inventors: Yoshihiro YAMADA, Kyoto-shi (JP); Kanae TERAMOTO, Kyoto-shi (JP)

(73) Assignee: SHIMADZU CORPORATION, Kyoto-shi (JP)

(21) Appl. No.: 17/685,453

(22) Filed: Mar. 3, 2022

(51) Int. Cl. G16B 40/00; H01J 49/00

(52) U.S. Cl. CPC G16B 40/00; H01J 49/0036

(57) ABSTRACT

To enable a correct and easy discrimination of microorganisms, a microorganism discrimination method includes: acquiring mass spectra related to known microorganisms which belong to the same species and whose subspecies, strains or types are known (S11); retrieving a list describing m/z values of marker-candidate proteins which are supposed to vary in mass among different subspecies, strains or types (S12); creating a mask which gives non-zero values only within a predetermined range including each of the listed m/z values (S14); masking each of the mass spectra (S15); creating wavelet images by performing continuous wavelet transform on the mass spectra (S16); creating a discriminant model by machine learning using, as training data, the wavelet images and information of the subspecies, strains or types of the known microorganisms; and discriminating the subspecies, strain or type of an unknown microorganism by applying a mass spectrum of this microorganism to the discriminant model.

### TECHNICAL FIELD

The present invention relates to a microorganism discrimination method and system.

### BACKGROUND ART

In recent years, a microorganism discrimination technique which employs matrix-assisted laser desorption/ionization mass spectrometry (MALDI-MS) has been rapidly spreading in clinical medicine, quality control and other related areas. In this technique, the discrimination of microorganisms is performed based on a mass spectrum obtained from a trace amount of a microorganism sample. The analysis result can be obtained within a short period of time. A continuous analysis for multiple specimens can also be easily performed. Thus, the technique enables a convenient and quick discrimination of microorganisms.

For the discrimination of microorganisms by MALDI-MS, it is necessary to locate a biomarker peak on the mass spectrum, i.e., a peak whose position and/or height varies among microorganisms that taxonomically belong to different groups (e.g., microorganisms which are of the same species yet belong to different strains), and to compare the biomarker peak on a mass spectrum of a microorganism to be discriminated with the biomarker peak on a mass spectrum of a known microorganism. In many cases, protein peaks are used as biomarker peaks for the discrimination of microorganisms, including bacilli. In particular, peaks of ribosomal proteins are often used (for example, see Non Patent Literatures 1 and 2).

### NON PATENT LITERATURE

Non Patent Literature 1: Kanae Teramoto, “Characterization of Bacteria by MALDI-MS”, Shimadu Review, Vol. 74, Nos. 1-2, Shimadzu Corporation, Sep. 20, 2017, pp. 51-62
Non Patent Literature 2: Kanae Teramoto and six other authors, “MALDI-MS Proteotyping of Cutibacterium acnes”, 2nd International BMS Symposium 2018 P-11, Oct. 26, 2018

### SUMMARY OF INVENTION

### Technical Problem

In particular, discriminating between closely related microorganisms (i.e., discrimination at the level of subspecies, strain or type) provides extremely useful information in medicine, food or other concerned areas for such purposes as the determination of the presence or absence of pathogenicity or identification of the source of infection. However, in the discrimination of microorganisms using a conventional MALDI-MS, discriminating between such closely related microorganisms requires checking a considerable number of biomarker peaks. There is room for further improvement in terms of the case of discrimination.

The present invention has been developed in view of the previously described point. Its objective is to provide a method, system and program for microorganism discrimination by which a highly accurate discrimination of microorganisms can be easily performed.

### Solution to Problem

A microorganism discrimination method according to the present invention developed for solving the previously described problem includes the steps of:
acquiring a plurality of mass spectra obtained by performing a mass spectrometric analysis on each of a plurality of known microorganisms which belong to the same species and whose subspecies, strains or types are known;
retrieving an m/z list describing m/z values of marker-candidate proteins each of which is supposed to vary in mass among different subspecies, different strains or different types in a group of microorganisms belonging to the same species as the known microorganisms;
creating a mask which gives non-zero values only within a predetermined m/z range including each m/z value described in the m/z list;
masking each of the plurality of mass spectra with the mask;
creating a plurality of wavelet images by performing continuous wavelet transform on each of the plurality of mass spectra after the masking;
creating a discriminant model by machine leaning using, as training data, the plurality of wavelet images and information of the subspecies, strain or type of each of the known microorganisms; and
discriminating the subspecies, strain or type of an unknown microorganism belonging to the same species as the known microorganisms, by applying, to the discriminant model, a mass spectrum acquired by performing a mass spectrometric analysis on the unknown microorganism whose subspecies, strain or type is unknown.

A microorganism discrimination system according to the present invention developed for solving the previously described problem includes:
a known-sample-data acquirer configured to acquire a plurality of mass spectra obtained by performing a mass spectrometric analysis on each of a plurality of known microorganisms which belong to the same species and whose subspecies, strains or types are known;
an m/z list retriever configured to retrieve an m/z list describing m/z values of marker-candidate proteins each of which is supposed to vary in mass among different subspecies, different strains or different types in a group of microorganisms belonging to the same species as the known microorganisms;
a mask creator configured to create a mask which gives non-zero values only within a predetermined m/z range including each m/z value described in the m/z list;
a masking processor configured to mask each of the plurality of mass spectra with the mask;
a wavelet image creator configured to create a plurality of wavelet images by performing continuous wavelet transform on each of the plurality of mass spectra after the masking;
a model creator configured to create a discriminant model by machine leaning using, as training data, the plurality of wavelet images and information of the subspecies, strain or type of each of the known microorganisms; and
a discriminator configured to discriminate the subspecies, strain or type of an unknown microorganism belonging to the same species as the known microorganisms, by applying, to the discriminant model, a mass spectrum acquired by performing a mass spectrometric analysis on the unknown microorganism whose subspecies, strain or type is unknown.

A program for microorganism discrimination according to the present invention developed for solving the previously described problem is a computer program for realizing the functions of the previously described microorganism discrimination system, and is configured to make a computer function as the components of the previously described microorganism discrimination system.

### Advantageous Effects of Invention

In the method, system and program for microorganism discrimination according to the present invention, a discriminant model for microorganism discrimination is created by machine learning based on mass spectra of a plurality of known microorganisms, and a mass spectrum of an unknown microorganism is applied to the discriminant model. By this technique, a highly accurate discrimination of microorganisms can be easily performed. For the creation of the discriminant model, the mass spectrum data of the known microorganisms are transformed into wavelet images which are two-dimensional images consisting of a plurality of pixels. Therefore, the data can be easily applied to a high-performance machine-learning algorithm, such as deep learning. The masking of the mass spectra before the transform into the wavelet images leads to the creation of wavelet images in which the differences between subspecies, strains or types are more noticeable. Accordingly, a discriminant model with an even higher level of discrimination capability can be created.

### BRIEF DESCRIPTION OF DRAWINGS

FIG. 1 is a schematic configuration diagram of a microorganism discrimination system according to one embodiment of the present invention.

FIG. 2 is a flowchart showing the flow of the processing in a training data creator.

FIG. 3 is a table showing one example of the m/z list in the present embodiment.

FIG. 4 is a graph showing one example of known sample data before and after calibration.

FIG. 5 is an entire view of a mask prepared based on the aforementioned m/z list.

FIG. 6 is an enlarged view of a section including m/z 6787 of the mask.

FIG. 7 is a graph showing a result obtained by applying the mask to the known sample data after calibration.

FIG. 8 is a chart showing one example of the wavelet image.

FIG. 9 is a chart showing a wavelet image obtained by extracting valid pixels from the wavelet image shown in FIG. 8.

FIG. 10 is a flowchart showing the flow of the processing in the discriminator.

FIG. 11 is a table showing the evaluation result of the discriminant model in an example.

### DESCRIPTION OF EMBODIMENTS

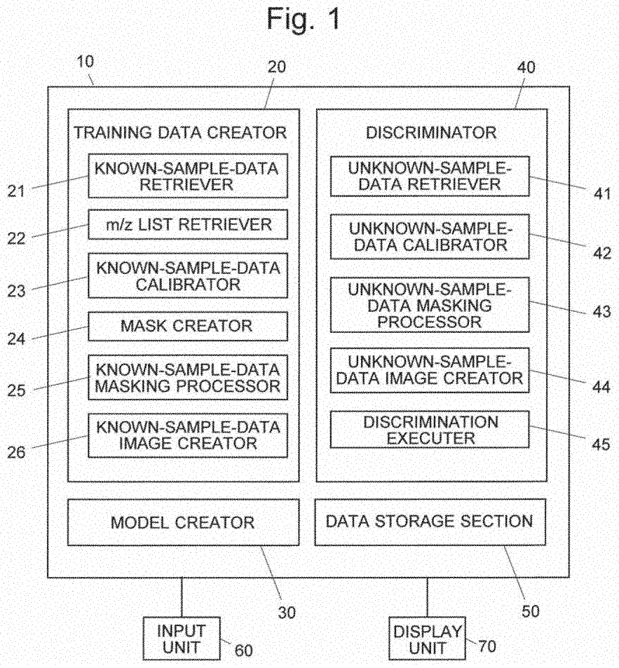

FIG. 1 is a diagram showing the configuration of the main components of a microorganism discrimination system 10 according to one embodiment of the present invention. This microorganism discrimination system 10 includes a training data creator 20, model creator 30, discriminator 40, data storage section 50, input unit 60 which includes a pointing device (e.g., mouse) and a keyboard (or similar device), as well as a display unit 70 which includes a display device (e.g., liquid crystal display).

The training data creator 20 creates training data to be used for machine learning, by performing a predetermined processing operation on mass spectrum data obtained by performing a MALDI-MS analysis on known microorganisms. The training data creator 20 includes a known-sample-data retriever 21, m/z list retriever 22, known-sample-data calibrator 23 (which corresponds to the calibrator in the present invention), mask creator 24, known-sample-data masking processor 25 (which corresponds to the masking processor in the present invention), and known-sample-data image creator 26 (which corresponds to the wavelet image creator in the present invention).

The model creator 30 creates a discriminant model for discriminating the subspecies, strain or type of unknown microorganisms, by a machine-learning algorithm using the training data.

The discriminator 40 discriminates the subspecies, strain or type to which an unknown microorganism belongs, by performing a predetermined processing operation on mass spectrum data obtained by a mass spectrometric analysis of the unknown microorganism and then applying the processed data to the discriminant model. The discriminator 40 includes an unknown-sample-data retriever 41, unknown-sample-data calibrator 42, unknown-sample-data masking processor 43, unknown-sample-data image creator 44, and discrimination executer 45.

The training data creator 20, model creator 30 and discriminator 40 are actually a personal computer or more sophisticated computer, on which the functions of the previously described components are realized by running, on the computer, dedicated data-analyzing software previously installed on the same computer. The data storage section 50 may be created on a storage device which is built in or directly connected to the computer. As another example, it is also possible to use a storage device located on a different computer system accessible from the aforementioned computer via the Internet (or the like), i.e., a storage device in cloud computing.

The microorganism discrimination system 10 according to the present embodiment can also be configured in such a manner that the functions of the training data creator 20, model creator 30 and discriminator 40 are assigned to a plurality of computers. Specifically, for example, it is possible to assign the functions of the training data creator 20 and the model creator 30 to one computer, and the function of the discriminator 40 to another computer.

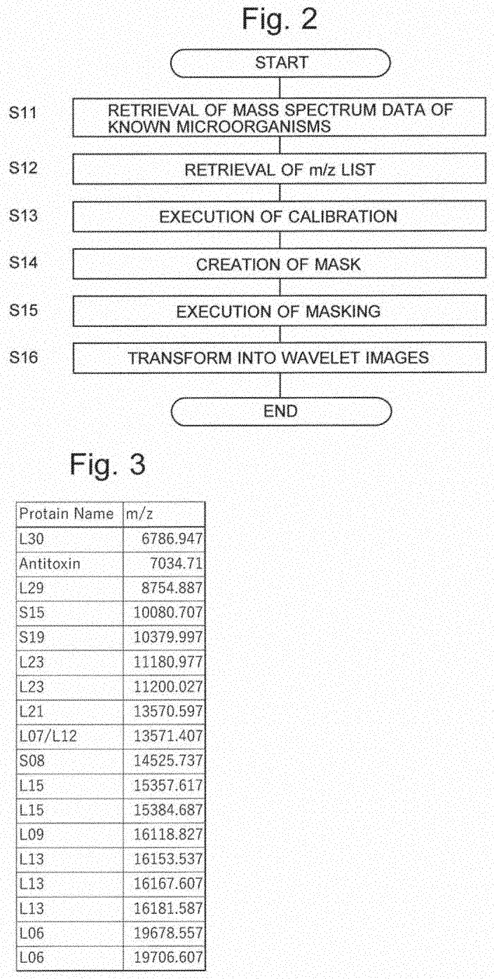

Details of the processing in the training data creator 20 will be hereinafter described with reference to the flowchart in FIG. 2.

Initially, the known-sample-data retriever 21 retrieves, from the data storage section 50, mass spectrum data of microorganisms whose subspecies, strains or types are known (Step S11). These microorganisms are hereinafter simply called the “known microorganisms”. The mass spectrum data of the known microorganisms are acquired beforehand by performing a MALDI-MS analysis on known microorganisms. Those data are previously stored in the storage section 50 and associated with the information of the subspecies, strains or types of the known microorganisms (this information is hereinafter called the “correct-answer label”).

Next, the m/z list retriever 22 retrieves, from the data storage section 50, a list which describes proteins each of which is supposed to vary in mass among the subspecies, strains or types of the microorganisms to be discriminated (these proteins are hereinafter called the “marker-candidate proteins”) as well as the m/z values of those proteins (this list is hereinafter called the “m/z list”; Step S12). The m/z list is prepared and stored in the data storage section 50 beforehand by the user or manufacturer of the microorganism discrimination system 10 according to the present embodiment. The marker-candidate proteins can be determined, for example, by comparing the base sequences or amino-acid sequences of a plurality of microorganisms belonging to different subspecies, strains or types, or by comparing mass spectra acquired by an actual MALDI-MS analysis of a plurality of microorganisms belonging to different subspecies, strains or types. The m/z value of each marker-candidate protein can be determined by converting the theoretical mass of each protein recorded in a public database, such as the NCBI (National Center for Biotechnology Information), into the m/z value of the ion originating from the protein concerned. For example, in the case of a MALDI-MS analysis of a microorganism sample prepared using a sinapinic acid as the matrix, the peak of the protonated molecule ([M+H]⁺) is most dominantly observed. Therefore, in this case, the conversion into the mass of the ion can be achieved by adding the mass of the proton to the theoretical mass of the marker-candidate protein. If the theoretical mass of a marker-candidate protein is not recorded in the public database, the theoretical mass may be calculated from the base sequence or amino-acid sequence of the marker-candidate protein, and the calculated mass may be converted into the m/z value of an ion to be included in the m/z list.

FIG. 3 shows one example of the m/z list created in Step S12. This list is an m/z list related to proteins which are supposed to vary in mass among different types of Cutibacterium acnes. The left column shows the names of the marker-candidate proteins, while the second column shows theoretical m/z values of those marker-candidate proteins. It should be noted that each protein for which two or more m/z values are shown in FIG. 3 (e.g., L23 or L15) is a protein for which it has been confirmed by a measurement that there is a variation in mass among the different types.

Subsequently, the known-sample-data calibrator 23 performs a calibration of the mass spectrum data of the known microorganisms retrieved in Step S11, using the m/z list retrieved in Step S12 (Step S13). Specifically, a peak detection is performed on the mass spectrum data of the known microorganisms to create a peak list (i.e., a list of the m/z values of the detected peaks). This peak list is compared with the m/z list retrieved in Step S11, and the horizontal axis of the mass spectrum data of the known microorganisms is corrected so as to cancel the discrepancy in the m/z value between the two lists.

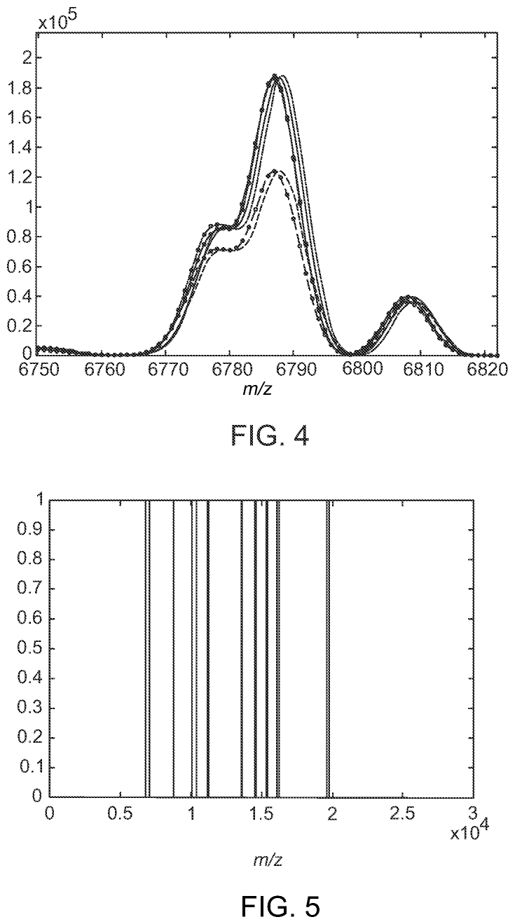

FIG. 4 shows one example of the result of a calibration of a mass spectrum using the m/z list shown in FIG. 3, where the mass spectrum was obtained by a MALDI-MS analysis of Cutibacterium acnes. It should be noted that FIG. 4 shows an enlarged view of a section including m/z 6787. In FIG. 4, the curves of the same line type (solid line, dashed line or chain line) show waveforms originating from the same sample, where the curve with no markers (white circles) is the waveform before the calibration, while the curve with the markers is the waveform after the calibration. As shown in FIG. 4, the position of the peak near m/z 6787 in the data before the calibration varies from one sample to another, while those peaks in the data after the calibration are aligned with the theoretical value (i.e., m/z 6787).

Subsequently, the mask creator 24 creates a mask which gives non-zero values only in the vicinity of the theoretical m/z value of each marker-candidate protein based on the m/z list (Step S14). Specifically, for example, the mask creator 24 prepares a virtual mass spectrum having a peak at each m/z value described in the m/z list retrieved in Step S12, and creates a mask which masks only the area over the border line formed by the waveform of the virtual mass spectrum. In this case, each peak in the virtual mass spectrum should preferably have the shape of a normal distribution. The height of each peak should typically exceed the saturation level. The value of the width of each peak should be appropriately determined taking into account an error in the occurrence position of the peak in MALDI-MS. The values of the height and width of the peak may be specified beforehand in the system. Alternatively, the system may be configured to allow the user to set those values as needed.

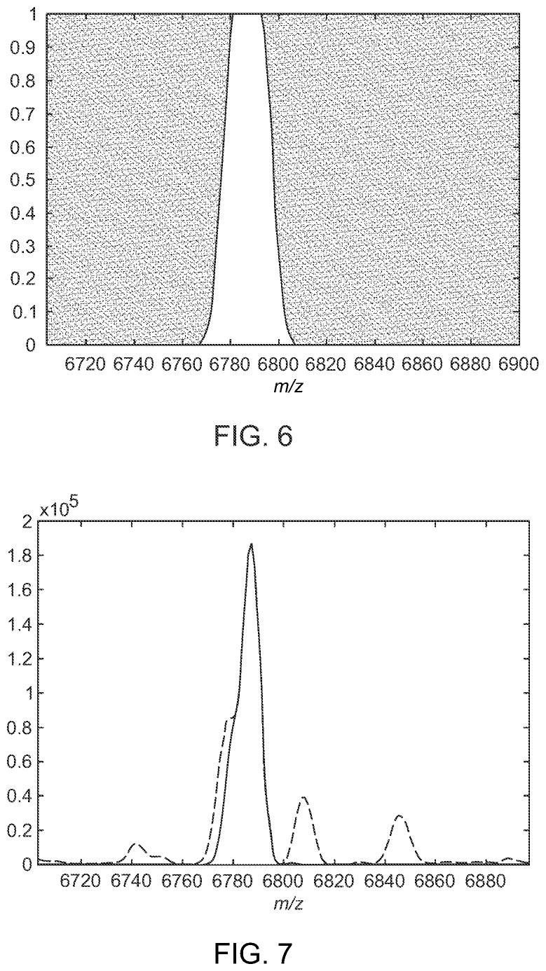

FIG. 5 shows one example of the mask created in Step S14. The mask in FIG. 5 has been created based on the m/z list in FIG. 3. This mask is designed to mask the areas except for the vicinity of each m/z value described in the m/z list. FIG. 6 shows an enlarged view of a section including m/z 6787 of the mask. In FIG. 6, the waveform of the peak around m/z 6787 forms the contour of the mask. The shaded areas correspond to the areas masked by this mask.

Next, the known-sample-data masking processor 25 performs the masking by applying the mask created in Step S14 to the mass spectrum data of the known microorganisms retrieved in Step S11 (Step S15). Consequently, each set of mass spectrum data becomes a mass spectrum which has values only in the vicinity of the theoretical m/z value of each marker-candidate protein.

FIG. 7 shows one example of the result obtained by masking a mass spectrum acquired by a MALDI-MS analysis of Cutibacterium acnes, using the mask shown in FIG. 5. It should be noted that FIG. 7 is an enlarged view of a section including m/z 6787 in the mass spectrum. The dashed line in FIG. 7 shows the data before the masking, while the solid line shows the data after the masking. As shown in the figure, the data after the masking have non-zero values only in the vicinity of the m/z value included in the m/z list (i.e., m/z 6787). Thus, peaks which are useless for the discrimination (such as the noise peaks, or peaks which show no significant variation among subspecies, strains or types) can be removed.

Subsequently, the known-sample-data image creator 26 converts the mass spectrum data of the known microorganisms after the masking (which are one dimensional signal data representing the correspondence relationship between m/z and intensity) into two-dimensional image data by continuous wavelet transform (Step S16). This data, which is hereinafter called the “wavelet image”, represents the signal intensity distribution after the wavelet transform in a graphical form, with the horizontal axis indicating the m/z value, the vertical axis indicating the frequency, and the pixel value indicating the signal intensity.

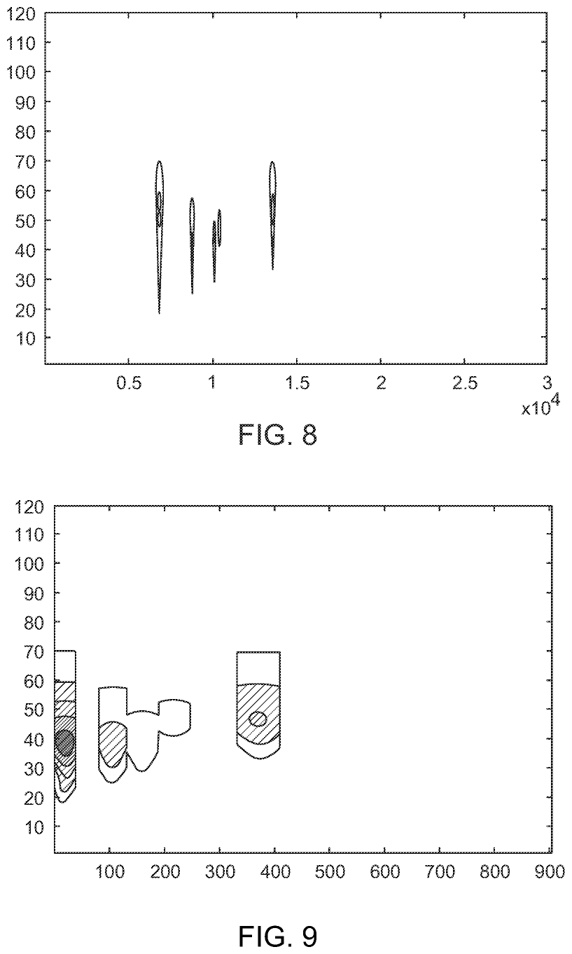

FIG. 8 shows one example of the result of the transform into a two-dimensional image (wavelet image) in which the aforementioned mass spectrum originating from Cutibacterium acnes was initially calibrated with the m/z list shown in FIG. 3 and then masked with the mask shown in FIG. 5 before being transformed. It should be noted that each pixel of the wavelet image is a complex number. In FIG. 8, the pixel values are converted into absolute values and represented by contour lines, and each area surrounded by a contour line is indicated by a different shading level (this note also applies in FIG. 9, which will be presented later).

As shown in FIG. 8, an image obtained by the wavelet transform includes a considerable amount of areas with small absolute values (i.e., white areas in the figure). Most of these areas originate from the areas masked in Step S15 and will not contribute to the discrimination of microorganisms. Therefore, it is preferable to remove pixels corresponding to these areas after the continuous wavelet transform, or to trim the no-value ranges off the mass spectrum before the continuous wavelet transform so as to maximally avoid the occurrence of such areas. FIG. 9 shows a wavelet image obtained by extracting valid pixels by the latter method.

The data storage section 50 holds a plurality of mass spectra originating from microorganisms of various subspecies, strains or types belonging to the same species. The training data creator 20 performs the previously described processing of Steps S11 through S16 for each of those mass spectra. A plurality of sets of wavelet image data thus obtained are stored in the data storage section 50 and respectively associated with the correct-answer label mentioned earlier.

Subsequently, the user operates the input unit 60 to issue a command to create a discriminant model using the plurality of sets of wavelet image data as the training data. Then, the model creator 30 begins to create a discriminant model (a mathematical model for microorganism discrimination). Specifically, the model creator 30 reads the wavelet images with the associated correct-answer labels from the data storage section 50 and creates a discriminant model by a predetermined type of machine-learning algorithm, using the read data and labels as the training data. As for the machine-learning algorithm, deep learning is typically used, although the available algorithm is not limited to it. Other types of machine-learning algorithms may also be used (e.g., support vector machine). The created discriminant model is stored in the data storage section 50 and associated with the m/z list created in Step S12 as well as the data of the mask created in Step S14.

At a later time, under the condition that a set of mass spectrum data obtained by a MALDI-MS analysis of an unknown microorganism to be discriminated has been stored in the data storage section 50, the user issues a command via the input unit 60 to perform a discrimination of the unknown microorganism by the discriminant model. Then, the discriminator 40 executes the discrimination process.

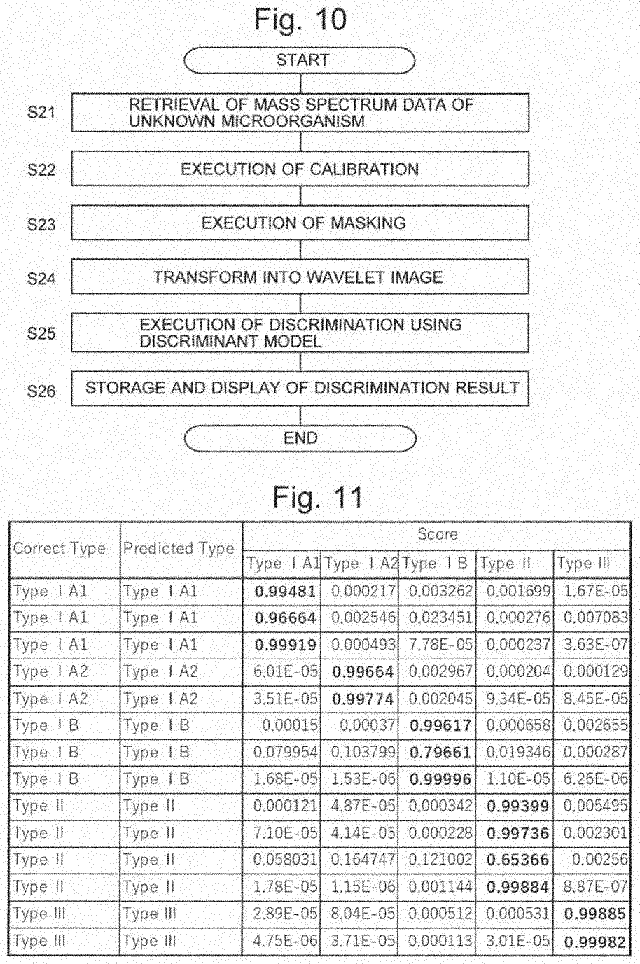

Details of the processing in the discriminator 40 are hereinafter described with reference to the flowchart of FIG. 10. Initially, the unknown-sample-data retriever 41 in the discriminator 40 retrieves, from the data storage section 50, the mass spectrum data of the unknown microorganism specified by the user (Step S21). Subsequently, the unknown-sample-data calibrator 42 reads the m/z list associated with the discriminant model in the data storage section 50 (i.e., the m/z list created in Step S12) and performs a calibration of the mass spectrum data of the unknown microorganism, using the m/z list (Step S22). Next the unknown-sample-data masking processor 43 reads the mask associated with the discriminant model in the data storage section 50 (i.e., the mask created in Step S14), and masks the mass spectrum after the calibration, using the mask (Step S23). Subsequently, the unknown-sample-data image creator 44 performs continuous wavelet transform of the mass spectrum data after the calibration and masking, to transform the mass spectrum into a wavelet image (Step S24). Details of the processing in Steps S22, S23 and S24 are similar to those of the processing in Steps S13, S15 and S16, respectively. Accordingly, descriptions of those steps will be omitted.

The discrimination executer 45 subsequently reads the discriminant model from the data storage section 50 and inputs the pixel values of the wavelet image data created in Step S24 into the discriminant model to determine, from the thereby obtained output values, what subspecies, strain or type the unknown microorganism belongs to (Step S25). The result of the discrimination by the discrimination executer 45 is stored in the data storage section 50. It is also displayed on the screen of the display unit 70 and presented to the user (Step S26).

A mode for carrying out the present invention has been described so far. It should be noted that the present invention is not limited to the previously described embodiment but can be appropriately changed or modified within the gist of the present invention. For example, in the previously described embodiment, the functions of the training data creator 20, model creator 30, and discriminator 40 are realized by one computer. These functions may be individually realized by separate computers. Additionally, in the previously described embodiment, both the known-sample-data retriever 21 and the unknown-sample-data retriever 41 are configured to retrieve the mass spectrum data of the known microorganisms and the correct-answer labels as well as the mass spectrum data of an unknown microorganism from the data storage section 50 created on a storage device in the computer on which these functional blocks are provided. Instead, these functional blocks may be configured to retrieve the known data and unknown data from another computer connected via a network.

### Example

One example of the microorganism discrimination method according to the present invention is hereinafter described. The present example is a case in which the present invention is applied to the typing (type discrimination) of Cutibacterium acnes. It should be noted that the present invention is also suitable for the typing, subspecies discrimination or strain discrimination of other kinds of microorganisms.

### 1. Acquisition of Known Sample Data

Cutibacterium acnes can be classified into five types (Type I A1, Type I A2, Type I B, Type II and Type III) according to their phenotypes, such as the morphology, constituents of the cell wall, and the result of a serotype agglutination test. In the present example, 45 strains of Cutibacterium acnes whose types were known were prepared as samples, and a MALDI-MS analysis was performed for each sample. From the 45 sets of mass spectrum data thus obtained, 70% of the mass spectrum data were randomly selected for the creation of the discriminant model. These mass spectra are hereinafter called the “training mass spectra”. The remaining 30% of the mass spectrum data were used for the evaluation of the discriminant model (as will be detailed later). These mass spectra are hereinafter called the “evaluation mass spectra”.

### 2. Creation of m/z List

Based on the amino-acid sequence information of Cutibacterium acnes obtained from NCBI, proteins each of which varies in mass among the types were extracted, from which some proteins that can be detected by MALDI-MS in a stable manner were further selected. Additionally, the theoretical masses of those proteins (marker-candidate proteins) were obtained from NCBI and converted into m/z values. Thus, an m/z list as shown in FIG. 3 was created.

### 3. Calibration

Using the m/z list, a calibration of the training mass spectra was performed. Specifically, for each of the training mass spectra, a peak detection was performed to create a peak list. Then, the peak list was compared with the m/z list, and the horizontal axis of each training mass spectrum was corrected so as to cancel the discrepancy in m/z value between the two lists, as shown in FIG. 4.

### 4. Masking

Based on the m/z list, a mask was created which gives non-zero values of the signal intensity only in the vicinity of each theoretical m/z value included in the m/z list. Using this mask, each training mass spectrum after the calibration was masked. The profile of the mask as well as the result obtained by applying the mask to a training mass spectrum were as illustrated in FIGS. 5-7.

5. Transform into Wavelet Image

After the areas having no values were removed from the training mass spectra which had undergone the calibration and masking, each mass spectrum was transformed into a wavelet image (as illustrated in FIG. 9) by continuous wavelet transform.

### 6. Creation of Discriminant Model

A discriminant model was created by deep learning, using, as the training data, the plurality of sets of wavelet image data obtained by performing the calibration, masking and transform into a wavelet image for each of the training mass spectra.

### 7. Evaluation of Discriminant Model

A test was conducted to determine whether or not the typing of the evaluation mass spectra could be correctly performed by the discriminant model created by the method described to this point. Specifically, for the 45 aforementioned sets of mass spectrum data obtained for the 45 known strains of Cutibacterium acnes whose types were known, the previously described steps of randomly dividing the sets of data into “training mass spectra” and “evaluation mass spectra”, creating a discriminant model using the “training mass spectra”, and typing the “evaluation mass spectra” by using the discriminant model were repeated 100 times, and the error rate for the discriminant model was calculated. As explained earlier, in the present example, the correct answers (i.e., the types of Cutibacterium acnes) for the “evaluation mass spectra” were also previously known. Therefore, it was possible to determine whether or not the typing of the “evaluation mass spectra” by the discriminant model was successful.

FIG. 11 shows one example of the result of the discrimination of the “evaluation mass spectra” performed by the discriminant model created by using the “training mass spectra”. In this example, all “evaluation mass spectra” were classified into the correct types. The 100-time repetition of the typing demonstrated that the error rate for the discriminant model in the present example was 6.7% on average. This result confirms that a satisfactory discriminant model can be created by the method according to the present invention.

[Various Modes]

A person skilled in the art can understand that the previously described illustrative embodiment is a specific example of the following modes of the present invention.

(Clause 1) A microorganism discrimination method according to one mode of the present invention includes the steps of:
acquiring a plurality of mass spectra obtained by performing a mass spectrometric analysis on each of a plurality of known microorganisms which belong to the same species and whose subspecies, strains or types are known;
retrieving an m/z list describing m/z values of marker-candidate proteins each of which is supposed to vary in mass among different subspecies, different strains or different types in a group of microorganisms belonging to the same species as the known microorganisms;
creating a mask which gives non-zero values only within a predetermined m/z range including each m/z value described in the m/z list;
masking each of the plurality of mass spectra with the mask;
creating a plurality of wavelet images by performing continuous wavelet transform on each of the plurality of mass spectra after the masking;
creating a discriminant model by machine leaning using, as training data, the plurality of wavelet images and information of the subspecies, strain or type of each of the known microorganisms; and
discriminating the subspecies, strain or type of an unknown microorganism belonging to the same species as the known microorganisms, by applying, to the discriminant model, a mass spectrum acquired by performing a mass spectrometric analysis on the unknown microorganism whose subspecies, strain or type is unknown.

(Clause 2) The microorganism discrimination method described in Clause 1 may further include the steps of:
comparing, for each of the mass spectra of the known microorganisms, the m/z value of a peak included in the mass spectrum with an m/z value described in the m/z list, and performing a calibration of the mass spectrum so as to reduce the difference between the two m/z values; and
performing the masking of the mass spectra after the calibration.

(Clause 3) The microorganism discrimination method described in Clause 1 or 2 may be configured as follows:
the known microorganisms are Cutibacterium acnes;
the marker-candidate proteins include ribosomal proteins L30, L29, S15, S19, L23, L21, L07/L12, S08, L15, L09, L13 and L06 as well as Antitoxin; and
the discriminating step is performed to discriminate the type of the unknown microorganism which is Cutibacterium acnes.

A microorganism discrimination system according to one mode of the present invention includes:
a known-sample-data acquirer configured to acquire a plurality of mass spectra obtained by performing a mass spectrometric analysis on each of a plurality of known microorganisms which belong to the same species and whose subspecies, strains or types are known;
an m/z list retriever configured to retrieve an m/z list describing m/z values of marker-candidate proteins each of which is supposed to vary in mass among different subspecies, different strains or different types in a group of microorganisms belonging to the same species as the known microorganisms;
a mask creator configured to create a mask which gives non-zero values only within a predetermined m/z range including each m/z value described in the m/z list;
a masking processor configured to mask each of the plurality of mass spectra with the mask:
a wavelet image creator configured to create a plurality of wavelet images by performing continuous wavelet transform on each of the plurality of mass spectra after the masking;
a model creator configured to create a discriminant model by machine leaning using, as training data, the plurality of wavelet images and information of the subspecies, strain or type of each of the known microorganisms; and
a discriminator configured to discriminate the subspecies, strain or type of an unknown microorganism belonging to the same species as the known microorganisms, by applying, to the discriminant model, a mass spectrum acquired by performing a mass spectrometric analysis on the unknown microorganism whose subspecies, strain or type is unknown.

(Clause 5) The microorganism discrimination system described in Clause 4 may further include a calibrator configured to compare, for each of the mass spectra of the known microorganisms, the m/z value of a peak included in the mass spectrum with an m/z value described in the m/z list, and to perform a calibration of the mass spectrum so as to reduce the difference between the two m/z values, where the masking by the masking processor is performed after the calibration of the mass spectra of the known microorganisms by the calibrator is performed.

(Clause 6) The microorganism discrimination system described in Clause 4 or 5 may be configured as follows:
the known microorganisms are Cutibacterium acnes;
the marker-candidate proteins include ribosomal proteins L30, L29, S15, S19, L23, L21, L07/L12, S08, L15, L09, L13 and L06 as well as Antitoxin; and
the discriminator is configured to discriminate the type of the unknown microorganism which is Cutibacterium acnes.

(Clause 7) A program for microorganism discrimination according to one mode of the present invention is configured to make a computer function as the components of the microorganism discrimination system described in one of claims 4-6.

In the method, system or program for microorganism discrimination described in Clause 1, 4 or 7, a discriminant model for microorganism discrimination is created by machine learning based on mass spectra of a plurality of known microorganisms, and a mass spectrum of an unknown microorganism is applied to the discriminant model. By this technique, a highly accurate discrimination of microorganisms can be easily performed. For the creation of the discriminant model, the mass spectrum data of the known microorganisms are converted into wavelet images. Therefore, the data can be easily applied to a high-performance machine-learning algorithm, such as the deep learning. The masking of the mass spectra before the conversion into the wavelet images leads to the creation of wavelet images in which the difference between subspecies, strains or types are more noticeable. Accordingly, a discriminant model with an even higher level of discrimination capability can be created.

In the microorganism discrimination method or system described in Clause 2 or 5, the mass spectra of the known microorganisms are calibrated before the transform of the mass spectra into wavelet images, whereby a discrepancy of the horizontal axis in the plurality of sets of mass spectrum data is corrected, so that the accuracy of the resulting discriminant model is improved.

By the microorganism discrimination method or system described in Clause 3 or 6, the typing of Cutibacterium acnes can be easily and accurately performed.

### REFERENCE SIGNS LIST

10 . . . Microorganism Discrimination System
20 . . . Training Data Creator
21 . . . Known-Sample-Data Retriever
22 . . . m/z List Retriever
23 . . . Known-Sample-Data Calibrator
24 . . . Mask Creator
25 . . . Known-Sample-Data Masking Processor
26 . . . Known-Sample-Data Image Creator
30 . . . Model Creator
40 . . . Discriminator
41 . . . Unknown-Sample-Data Retriever
42 . . . Unknown-Sample-Data Calibrator
43 . . . Unknown-Sample-Data Masking Processor
44 . . . Unknown-Sample-Data Image Creator
45 . . . Discrimination Executer
50 . . . Data Storage Section
60 . . . Input Unit
70 . . . Display Unit

1. A microorganism discrimination method, comprising steps of:
acquiring a plurality of mass spectra obtained by performing a mass spectrometric analysis on each of a plurality of known microorganisms which belong to a same species and whose subspecies, strains or types are known;
retrieving an m/z list describing m/z values of marker-candidate proteins each of which is supposed to vary in mass among different subspecies, different strains or different types in a group of microorganisms belonging to the same species as the known microorganisms;
creating a mask which gives non-zero values only within a predetermined m/z range including each m/z value described in the m/z list;
masking each of the plurality of mass spectra with the mask;
creating a plurality of wavelet images by performing continuous wavelet transform on each of the plurality of mass spectra after the masking;
creating a discriminant model by machine leaning using, as training data, the plurality of wavelet images and information of the subspecies, strain or type of each of the known microorganisms; and
discriminating the subspecies, strain or type of an unknown microorganism belonging to the same species as the known microorganisms, by applying, to the discriminant model, a mass spectrum acquired by performing a mass spectrometric analysis on the unknown microorganism whose subspecies, strain or type is unknown.

2. The microorganism discrimination method according to claim 1, further including steps of:
comparing, for each of the plurality of mass spectra, a m/z value of a peak included in the mass spectrum with an m/z value described in the m/z list, and performing a calibration of each of the plurality of mass spectra so as to reduce a difference between the two m/z values; and
performing the masking of each of the plurality of mass spectra after the calibration.

3. The microorganism discrimination method according to claim 1, wherein:
each of the plurality of known microorganisms is Cutibacterium acnes;
the marker-candidate proteins include ribosomal proteins L30, L29, S15, S19, L23, L21, L07/L12, S08, L15, L09, L13 and L06 as well as Antitoxin; and
the discriminating step is performed to discriminate the type of the unknown microorganism which is Cutibacterium acnes.

4. A microorganism discrimination system, comprising:
a known-sample-data acquirer configured to acquire a plurality of mass spectra obtained by performing a mass spectrometric analysis on each of a plurality of known microorganisms which belong to a same species and whose subspecies, strains or types are known;
an m/z list retriever configured to retrieve an m/z list describing m/z values of marker-candidate proteins each of which is supposed to vary in mass among different subspecies, different strains or different types in a group of microorganisms belonging to the same species as the known microorganisms;
a mask creator configured to create a mask which gives non-zero values only within a predetermined m/z range including each m/z value described in the m/z list;
a masking processor configured to mask each of the plurality of mass spectra with the mask;
a wavelet image creator configured to create a plurality of wavelet images by performing continuous wavelet transform on each of the plurality of mass spectra after the masking;
a model creator configured to create a discriminant model by machine leaning using, as training data, the plurality of wavelet images and information of the subspecies, strain or type of each of the known microorganisms; and
a discriminator configured to discriminate the subspecies, strain or type of an unknown microorganism belonging to the same species as the known microorganisms, by applying, to the discriminant model, a mass spectrum acquired by performing a mass spectrometric analysis on the unknown microorganism whose subspecies, strain or type is unknown.

5. The microorganism discrimination system according to claim 4, further comprising a calibrator configured to compare, for each of the plurality of mass spectra, a m/z value of a peak included in the mass spectrum with an m/z value described in the m/z list, and to perform a calibration of each of the plurality of mass spectra so as to reduce a difference between the two m/z values,
wherein the masking by the masking processor is performed after the calibration of each of the plurality of mass spectra by the calibrator is performed.

6. The microorganism discrimination system according to claim 4, wherein:
each of the plurality of known microorganisms is Cutibacterium acnes;
the marker-candidate proteins include ribosomal proteins L30, L29, S15, S19, L23, L21, L07/L12, S08, L15, L09, L13 and L06 as well as Antitoxin; and
the discriminator is configured to discriminate the type of the unknown microorganism which is Cutibacterium acnes.

7. A non-transitory computer readable medium recording a microorganism discrimination program configured to make a computer function as components of the microorganism discrimination system according to claim 4.
